(19) United States
(12) Patent Application Publication
(10) Pub. No.: US 2013/0006384 A1
(43) Pub. Date: Jan. 3, 2013

(54) CULTURE MEDIUM, GRAFT, AND MANUFACTURING METHOD THEREOF

(75) Inventors: CHEN FENG, Beijing (CN); LI FAN, Beijing (CN); WEN-MEI ZHAO, Beijing (CN)

(73) Assignees: HON HAI PRECISION INDUSTRY CO., LTD., Tu-Cheng (TW); TSINGHUA UNIVERSITY, Beijing (CN)

(21) Appl. No.: 13/484,593

(22) Filed: May 31, 2012

(30) Foreign Application Priority Data
Jun. 30, 2011 (CN) 201110181525.6

(51) Int. Cl. A61F 2/02; C12N 5/02; B82Y 5/00

(52) U.S. Cl. 623/23.72; 435/395; 435/404; 977/742; 977/923

(57) ABSTRACT

A graft includes a carbon nanotube structure and a biological tissue. The carbon nanotube structure has a polar surface. The polar surface is formed by treating the carbon nanotube structure with polarization. The biological tissue is adhered on the polar surface. In addition, a method for manufacturing a graft is also provided.

This application claims all benefits accruing under 35 U.S.C. §119 from China Patent Application No. 201110181525.6, filed on Jun. 30 2011 in the China Intellectual Property Office, disclosure of which is incorporated herein by reference.

### BACKGROUND

1. Technical Field

The present disclosure relates to a culture medium, a graft, and a method for manufacturing a graft.

2. Description of Related Art

Many people suffer from neurological disorders as a result of neuron injuries. Neural grafting can provide relief from these injuries. Neural grafting is a surgical transfer of tissue from various sources into specific areas of the nervous system that have been affected by injury. The neural grafting serves as a “bridge” to connect the proximate injured neurons. Grafted cells may synthesize and release growth-promoting factors near the injured neurons, thereby promoting neuron regeneration.

What is needed, therefore, is to provide a culture medium, a graft, and a method for manufacturing the graft to be employed in wound to recover promptly.

### BRIEF DESCRIPTION OF THE DRAWINGS

Many aspects of the disclosure can be better understood with reference to the drawings. The components in the drawings are not necessarily drawn to scale, the emphasis instead being placed upon clearly illustrating the principles of the present disclosure. Moreover, in the drawings, like reference numerals designate corresponding parts throughout the several views.

FIG. 1 is a schematic view of one embodiment of a graft.

FIG. 2 is a scanning electron microscope (SEM) image of one embodiment of a drawn CNT film.

FIG. 3 is a SEM image of one embodiment of stacked CNT films.

FIG. 4 is a SEM image of one embodiment of a pressed CNT film.

FIG. 5 is a SEM image of one embodiment of a flocculated CNT film.

FIG. 6 is a SEM image of one embodiment of an untwisted carbon nanotube wire.

FIG. 7 is a SEM image of one embodiment of a twisted carbon nanotube wire.

FIG. 8 is a schematic view of one embodiment of a graft with bearing.

FIG. 9 shows a schematic view of a process for manufacturing the graft of FIG. 1.

FIG. 10 is an optical microscopy image of one embodiment of differentiated neurons stained with fluorescence.

FIG. 11 is an optical microscopy image of another embodiment of differentiated neurons stained with fluorescence.

### DETAILED DESCRIPTION

The disclosure is illustrated by way of example and not by way of limitation in the figures of the accompanying drawings in which like references indicate similar elements. It should be noted that references to “an” or “one” embodiment in this disclosure are not necessarily to the same embodiment, and such references mean at least one.

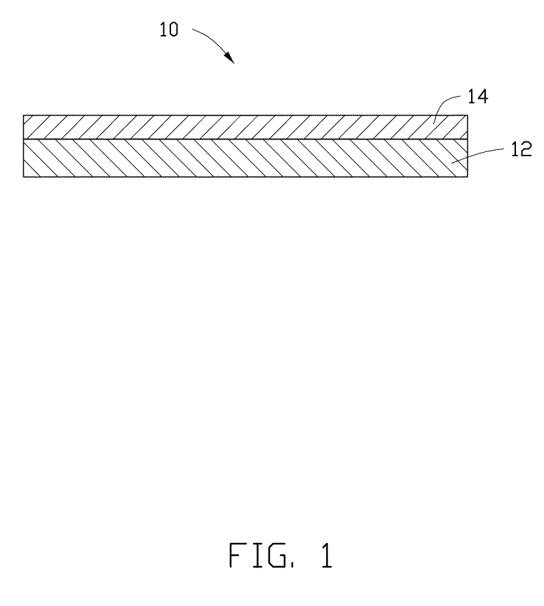

Referring to FIG. 1, one embodiment of a graft 10 includes a carbon nanotube (CNT) structure 12 and a biological tissue 14. The CNT structure 12 has a polar surface. The biological tissue 14 is adhered on the CNT structure 12. Particularly, the biological tissue 14 is cultured on the polar surface of the CNT structure 12. In the present embodiment, the biological tissue 14 can be an ensemble of a variety of cells. The cells of the present embodiment can be, but not limited to, neuronal cells, skin cells, or muscle cells.

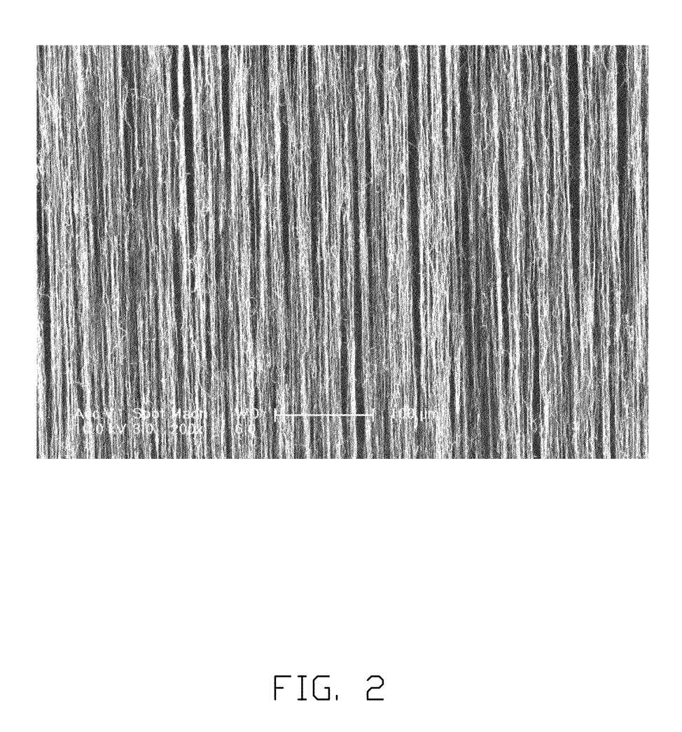

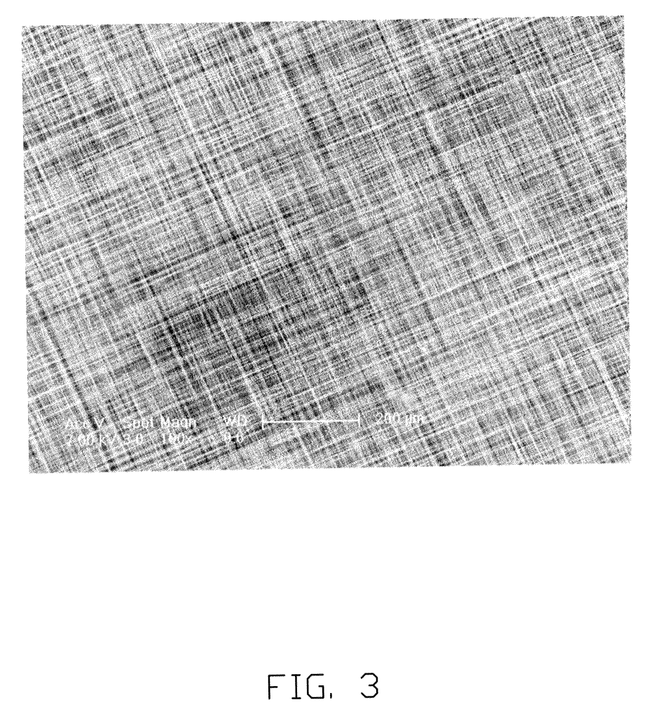

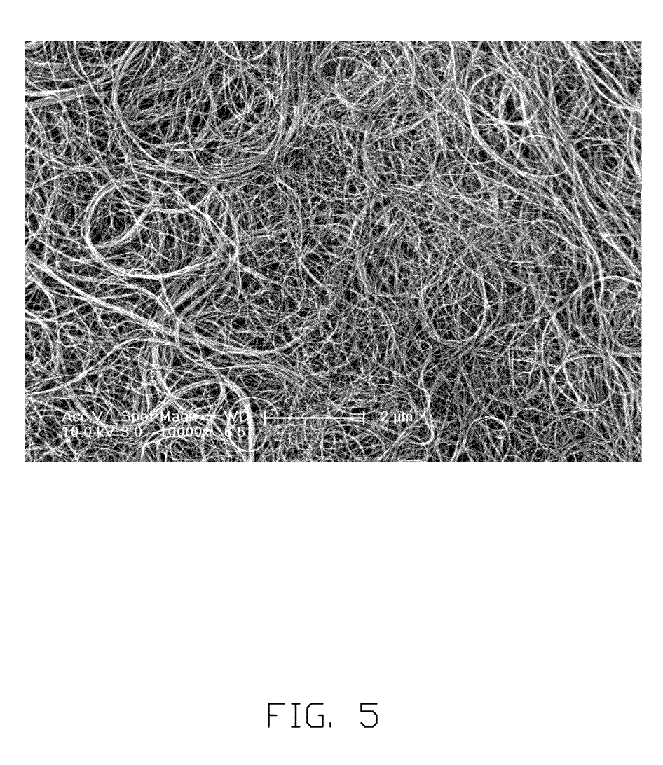

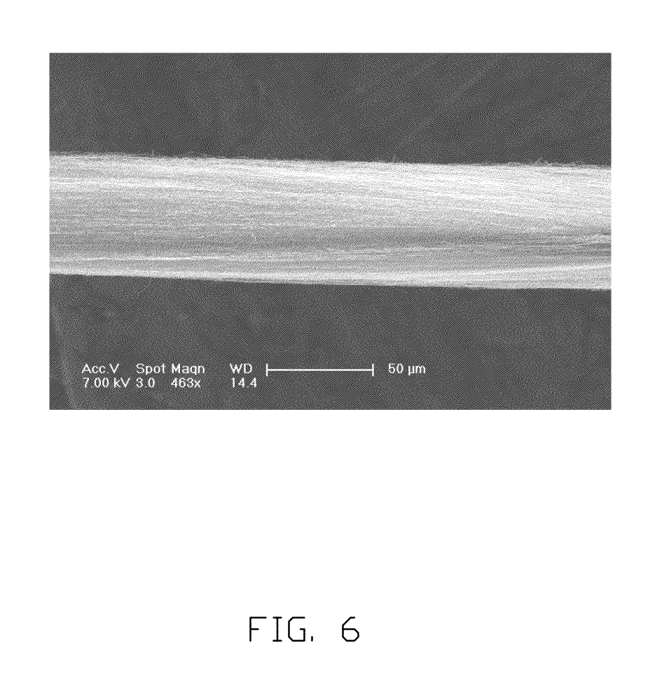

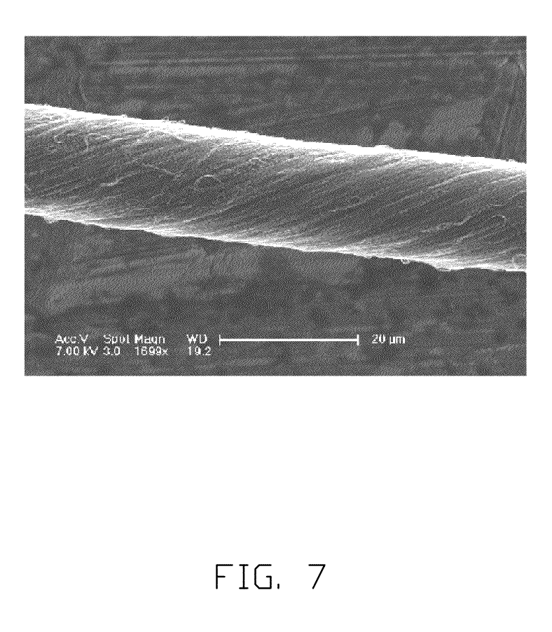

The CNT structure 12 includes a plurality of carbon nanotubes to be arranged in a shape of a film or a wire. Specifically, the carbon nanotubes can be aligned to form a CNT film, as shown in FIG. 2, FIG. 3, and FIG. 5, or a CNT wire, as shown in FIG. 6 and FIG. 7. Whether the CNT structure 12 is made up by CNT films or wires, the carbon nanotubes are connected by van der Waals attractive force. Consequentially, the CNT structure 12 is a free-standing structure. That is, the CNT structure 12 of the present embodiment can retain a specific shape without any supporter because of the strong connections by van der Waals attractive force between adjacent carbon nanotubes. In addition, the CNT structure 12 connected by van der Waals attractive force can bend easily without breaking. Thus, the graft 10 of the present embodiment has good elasticity and ductility, so that the graft 10 can be tailored arbitrarily and employed to cure an area with any shape.

Referring to FIG. 2, the CNT film can be formed by drawing carbon nanotubes from a CNT array grown on a silicon wafer by vapor deposition. Accordingly, a width of the CNT film corresponds to a size of the CNT array. The drawn carbon nanotubes are also joined end to end in succession by van der Waals attractive force and are orientated substantially in the same direction.

Referring to FIG. 3, the CNT structure 12 can include two or more CNT films stacked with each other. The adjacent CNT films are connected to each other by van der Waals attractive force. A thickness of the CNT structure 12 is arbitrary. The thickness of the CNT structure and the layers of the stacked CNT films are directly proportional. In the present embodiment, the CNT structure 12 is comprised of ten layers of CNT films.

The CNT films can be stacked in succession such that adjacent CNT films can intersect with each other. Thus, the mechanical strength of the CNT structure 12 is enhanced. An existing angle between two adjacent CNT films can be in a range from about 0 degrees to about 90 degrees. If the existing angle is more than 0 degrees, the CNT films intersect to form a mesh-like structure. In the present embodiment, the CNT films of the CNT structure 12 form a mesh-like structure with carbon nanotubes intersecting substantially perpendicular to each other. The number of carbon nanotubes films and the angles made by adjacent CNT films are arbitrary and are set according to practical requirements.

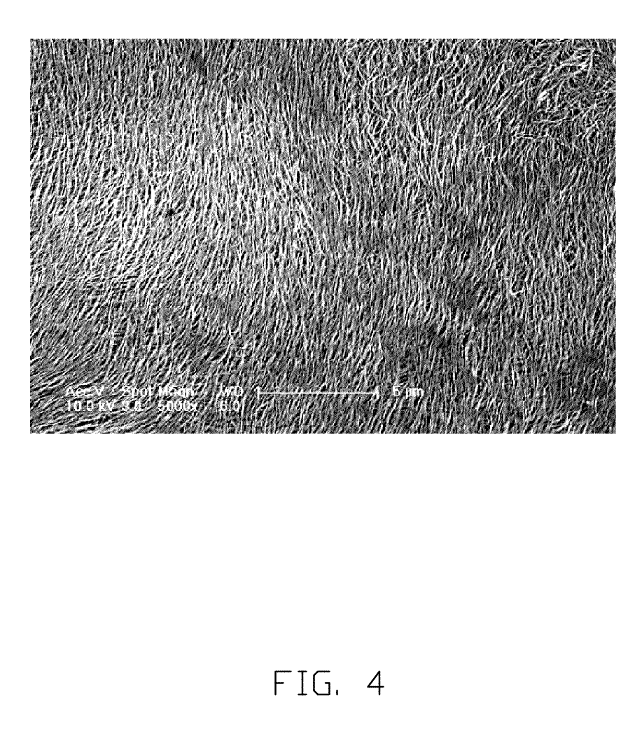

Referring to FIG. 4, the CNT film can also be formed by pressing the CNT array. The carbon nanotubes of the CNT film are pressed to lay partially over each other. In the present embodiment, the length of the carbon nanotubes of the pressed CNT film is more than 45 μm. In this case, a plurality of micropores or interstitial spaces are defined in the pressed CNT film. A dimension of the micropore or interstitial space is in a range from about 1 nm to about 450 nm. If the carbon nanotube array is pressed along a variety of directions, the carbon nanotubes of the CNT film are oriented in different directions. If the carbon nanotube array is pressed toward one direction, the carbon nanotubes of the CNT films are primarily oriented in that one direction. The carbon nanotubes of the pressed CNT film are connected to each other by van der Waals attractive force. Thus, the CNT film can be a free-standing structure and can be bent arbitrarily. An angle of the carbon nanotubes and a surface of the CNT film ranges from about 0 degrees to about 15 degrees. Preferably, the carbon nanotubes of the pressed CNT film are substantially parallel to the surface of the CNT film. In addition, a thickness of the CNT film is closely related to the height of the carbon nanotube array, and the pressure applied to the carbon nanotube array. In the present embodiment, a thickness of the CNT film is in a range from about 0.5 nm to about 100 μm.

Referring to FIG. 5, the CNT film can also be obtained by flocculating the carbon nanotube array. In this case, the carbon nanotubes in the CNT film are entangled by the van der Waals attractive force therebetween, thereby allowing the CNT film to form a microporous structure. The CNT film has a plurality of micropores with diameters in a range from about 1 nm to about 450 nm. In the present embodiment, a length of the carbon nanotube is more than 10 μm. Preferably, the length of carbon nanotube is in a range from about 200 μm to about 900 μm. The length of the carbon nanotubes should be long enough to entangle with each other.

Referring to FIG. 6 and FIG. 7, a CNT structure 12 in accordance with another embodiment can comprise a plurality of CNT wires. Each of the CNT wires may be formed by bundling a plurality of carbon nanotubes together. The carbon nanotubes are substantially parallel to an axis of the CNT wire to form a bundled CNT wire. The bundled CNT wire can be obtained by treating the drawn CNT film with an organic solvent, such as ethanol, methanol, acetone, dichloroethane, or chloroform. In addition, each of the CNT wires can be formed by bundling and twisting a plurality of carbon nanotubes together. The carbon nanotubes are aligned helically around an axis of the CNT wire to form a twisted CNT wire. In practice, the twisted CNT wire can be obtained by either twisting the bundled CNT wire or twisting the CNT film via a mechanical force. As mentioned previously, the bundled or twisted CNT wire has a diameter ranging from about 0.5 nm to about 1 mm.

In the present embodiment, the carbon nanotubes of the CNT structure 12 are treated by polarization to form the polar surface on the CNT structure 12. The polar surface provides an environment to culture grafted cells. The grafted cells can adhere strongly on the polar surface of the CNT structure 12 because a polarity of the polar surface attracts an opposite polarity of the grafted cells, forming the biological tissue 14 on the CNT structure 12. In the present embodiment, the polar surface of the CNT structure 12 is formed by treating the carbon nanotubes with poly-D-lysine solution or polyetherimide solution.

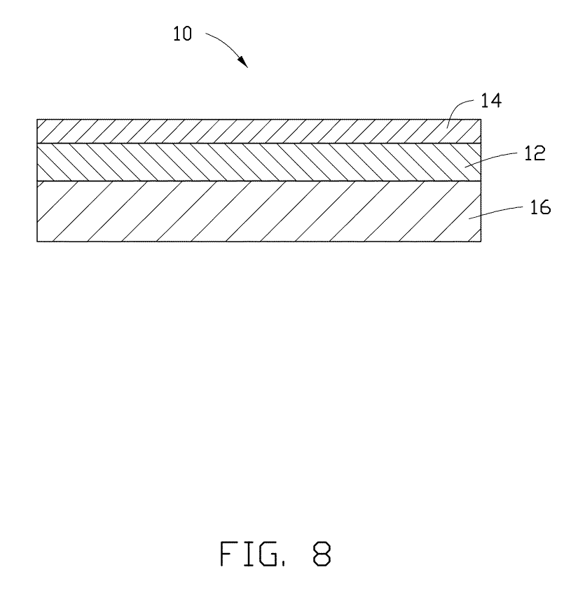

In addition, referring to FIG. 8, the graft 10 of the present embodiment can further comprise a bearing 16 to support the CNT structure 12. In such case, the CNT structure 12 is located on the bearing 16 and the biological tissue 14 is opposite the bearing 16. The bearing 16 can be made of biodegradable or non-biotoxic material, forming a biocompatible graft to be used in a biological environment. For example, the biodegradable material can be thermoplastic starch, polylactide, polyvinyl alcohol, or aliphatic polyesters. The non-biotoxic material can be silicon. In the present embodiment, the bearing 16 can be tailored according to the thickness and the dimension of the CNT structure 12. Because the graft 10 with bearing 16 has better mechanical strength than the graft 10 without bearing 16, the graft 10 of the present embodiment can be applied to any irregular-shaped wounded area. In addition, the bearing 16 employed in a biological environment does not need to be removed as it is biodegradable.

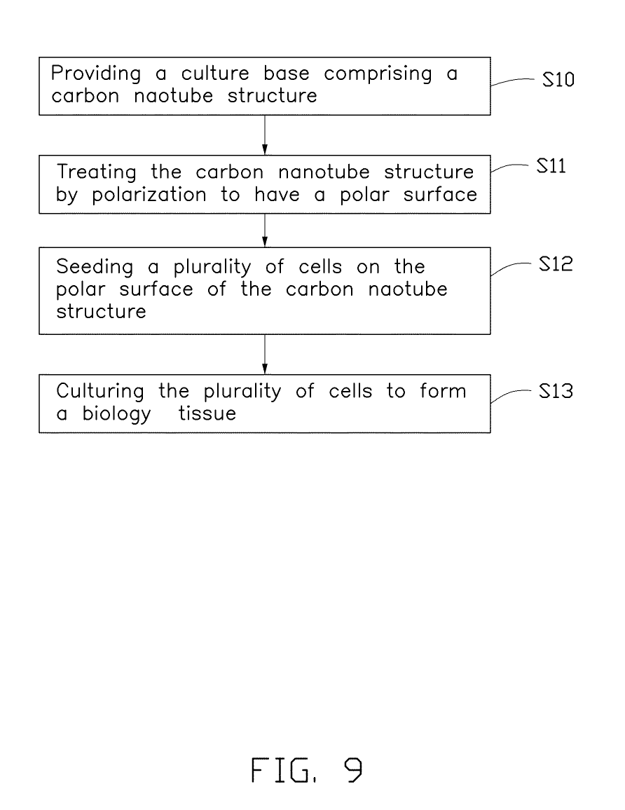

Referring to FIG. 9, a method for manufacturing a graft includes:

S10, providing a culture base comprising a CNT structure 12;

S11, treating the CNT structure 12 by polarization to have a polar surface;

S12, seeding a plurality of cells on the polar surface of the CNT structure 12; and

S13, culturing the plurality of cells to form a biological tissue 14. In the present embodiment, the cells can be neuronal cells, skin cells, or muscle cells.

The method is described in more detail as follows.

In step S10, the CNT structure 12 is formed by one or more CNT films or CNT wires.

In Step S11, the CNT structure 12 can be sterilized in advance. In the present embodiment, the CNT structure 12 is sterilized by irradiating or heating. For example, the CNT structure 12 can be irradiated with ultraviolet light for about 0.5 hours or heated at about 120° C. to eliminate microorganisms, such as bacteria. The sterilized CNT structure 12 is then treated with a poly-D-lysine solution or polyetherimide solution to form a polar surface on the CNT structure 12. In the present embodiment, the sterilized CNT structure 12 is soaked in the polyetherimide solution with a concentration of about 20 μg/ml. After immersing the CNT structure 12 in the poly-D-lysine solution or polyetherimide solution, the poly-D-lysine or polyetherimide coating is removed by rinsing the CNT structure 12 with deionized water immediately. In such case, treatment of the poly-D-lysine solution or polyetherimide solution will electrically charge the CNT structure 12, enhancing the cells attachment and adhesion. Polarizing the CNT structure 12 of the present embodiment is a simple way to achieve cell attraction compared to coating, sputtering or functionalizing the CNT structure 12 to provide cell adhesion environment.

To strengthen the CNT structure 12, the culture base further comprises a bearing 16 where the CNT structure 12 with the polar surface is located. In such case, the polar surface is opposite to the bearing 16. Specifically, the polar surface is far away from the bearing 16. In the present embodiment, the material, shape, or thickness of the bearing 16 can be chosen according to the practice need. For example, the bearing 16 can be a flat or curved structure. In the present embodiment, the bearing 16 is biodegradable and non-biotoxic. In such case, the graft 10 of the present embodiment is biocompatible when employed in the biological environment. Alternatively, the bearing 16 can be a culture dish where the CNT structure 12 is located and the cells are cultured.

In addition, an organic solution can be dropped on the CNT structure 12 to enhance the adhesion between the CNT structure 12 and the bearing 16. As a result, the specific surface area of the CNT structure 12 is reduced by evaporating the organic solution, and the adhesion between the CNT structure 12 and the bearing 16 is enhanced. In the present embodiment, ethyl alcohol is dropped on and evaporated from the CNT structure 12 to enhance the adhesion between the CNT structure 12 and the bearing 16.

As mentioned above, when the culture base with bearing 16 is treated to have a polar surface on the CNT structure 12, the culture base with bearing 16 is also sterilized by irradiating or heating in advance. The CNT structure 12 and the bearing 16 are irradiated for about 0.5 hours and heated at about 120° C. for about 20 minutes. The poly-D-lysine solution or polyetherimide solution is then employed on the sterilized CNT structure 12 to polarize the CNT structure 12.

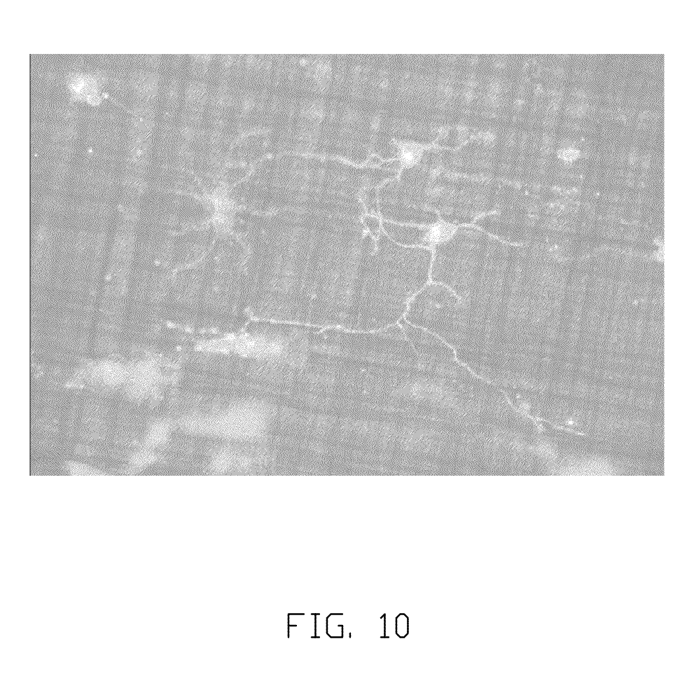

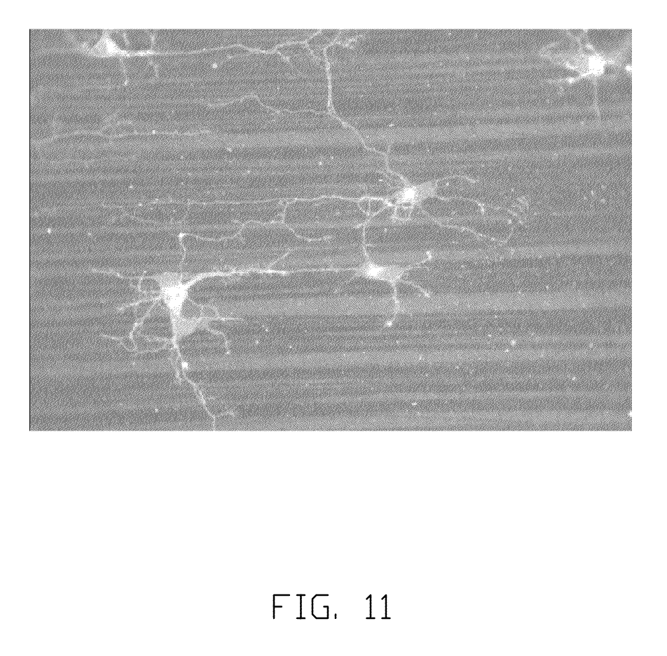

In the present embodiment, a variety of cells can be seeded on the culture base, particularly on the polar surface of the CNT structure. Referring to FIG. 10 and FIG. 11, neuronal cells (e.g. hippocampal neurons) are used as an example. The difference between FIG. 10 and FIG. 11 is the number of layers of CNT films used in the CNT structure. Specifically, the CNT structure 12 with ten layers of CNT film used to culture neuronal cells is shown in FIG. 10 while the CNT structure 12 with one layer of CNT film used to culture neuronal cells is shown in FIG. 11. In the present embodiment, the culture base is placed in a cell culture dish to culture the cells. The cell growth factor chosen according to the type of cells to be cultured is necessary to nourish cells growth and differentiate a variety of cell types. In the present embodiment, the hippocampal neurons on the culture base are incubated in an incubator with a CO₂ concentration of about 5% and a temperature of about 37° C. During the optimal culture period, a plurality of neuritis from a cell body of the individual neuron cells will branch toward and connect with adjacent neurons to form a neuronal network. Thus, the neuronal communication can be propagated. The neurons on the culture base of the present embodiment are connected to each other after incubating for about 7 days.

In the present embodiment, due to the flexibility of the CNT structure 12, the graft 10 can be tailored arbitrarily and employed to cure an area with any shape. In addition, the polar surface of CNT structure 12 provides an environment for cell adhesion and cell growth. Thus, the graft 10 of the present embodiment uses a simple way to attract cells and have a wide range of applications in the biological environment.

It is to be understood that the above-described embodiments are intended to illustrate rather than limit the disclosure. Any elements described in accordance with any embodiments is understood that they can be used in addition or substituted in other embodiments. Embodiments can also be used together. Variations may be made to the embodiments without departing from the spirit of the disclosure. The above-described embodiments illustrate the scope of the disclosure but do not restrict the scope of the disclosure.

Depending on the embodiment, certain of the steps of methods described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

What is claimed is:

1. A graft, comprising:
a carbon nanotube structure having a polar surface; and
a biological tissue adhered on the polar surface.

2. The graft of claim 1, wherein the polar surface is formed by treating the carbon nanotube structure with poly-D-lysine solution or polyetherimide solution.

3. The graft of claim 1, wherein the carbon nanotube structure is a free-standing structure comprising a plurality of carbon nanotubes connected by van der Waals attractive force.

4. The graft of claim 3, wherein the carbon nanotubes are polarized to have a polarity, which attracts the biological tissue.

5. The graft of claim 1, further comprising a bearing, wherein the carbon nanotube structure is located between the bearing and the biological tissue.

6. The graft of claim 5, wherein the bearing is biodegradable and non-biotoxic.

7. The graft of claim 1, wherein the biological tissue is skin tissue, neuronal tissue, or muscle tissue.

8. A method for manufacturing a graft, comprising:
providing a culture base comprising a carbon nanotube structure;
treating the carbon nanotube structure by polarization to have a polar surface;
seeding a plurality of cells on the polar surface of the carbon nanotube structure; and
culturing the plurality of cells to form a biological tissue.

9. The method of claim 8, wherein the carbon nanotube structure comprises at least one carbon nanotube film.

10. The method of claim 9, wherein the at least one carbon nanotube film comprises a plurality of successive and oriented carbon nanotubes joined end to end by van der Waals attractive force, and the at least one carbon nanotube film is either formed by a plurality of carbon nanotubes entangled with each other or a plurality of carbon nanotubes arranged isotropically.

11. The method of claim 9, wherein the carbon nanotube structure comprises a plurality of carbon nanotube films stacked together, and adjacent carbon nanotube films are combined and attracted to each other only by van der Waals attractive force therebetween.

12. The method of claim 8, wherein the carbon nanotube structure comprises a plurality of carbon nanotube wires, and one of the carbon nanotube wires is in a twisted form.

13. The method of claim 8, wherein the step of treating the carbon nanotube structure by polarization comprises:
sterilizing the carbon nanotube structure; and
employing a poly-D-lysine solution or polyetherimide solution on the sterilized carbon nanotube structure.

14. The method of claim 13, further comprising a step of removing the poly-D-lysine solution or polyetherimide solution from the carbon nanotube structure.

15. The method of claim 8, wherein the culture base further comprises a bearing and the carbon nanotube structure is located on the bearing.

16. The method of claim 15, wherein the step of treating the carbon nanotube structure by polarization comprises:
sterilizing the carbon nanotube structure and the bearing; and
employing a poly-D-lysine solution or polyetherimide solution on the sterilized carbon nanotube structure.

17. A culture medium for culturing at least one kind of cell tissue, the culture medium comprising a carbon nanotube structure having a polar surface with a polarity attracting an opposite polarity of the cell tissue.

18. The culture medium of claim 17, wherein the carbon nanotube structure comprises a plurality of carbon nanotubes connected by van der Waals attractive force, and the carbon nanotubes are polarized to form the polar surface.

19. The culture medium of claim 17, further comprising a bearing, wherein the carbon nanotube structure is located on one surface of the bearing, and the polar surface of the carbon nanotube structure is away from the bearing.

20. The culture medium of claim 19, wherein the bearing is biodegradable and non-biotoxic.
